(19) United States
(12) Patent Application Publication
(10) Pub. No.: US 2015/0020812 A1
(43) Pub. Date: Jan. 22, 2015

(54) SLEEP APPLIANCE WITH OXYGEN

(71) Applicants: Bryan KEROPIAN, Woodland hills, CA (US); Brian SPRIGGS, Everett, WA (US)

(72) Inventors: Bryan Keropian, Woodland Hills, CA (US); Brian Spriggs, Everett, WA (US)

(21) Appl. No.: 14/382,891

(22) PCT Filed: Mar. 5, 2013
(86) PCT No.: PCT/US13/29067

§ 371 Date: Sep. 4, 2014

Related U.S. Application Data
(60) Provisional application No. 61/607,720, filed on Mar. 7, 2012.

(51) Int. Cl. A61F 5/56; A61M 16/10; A61M 16/08; A61M 16/04

(52) U.S. Cl. CPC A61F 5/566; A61M 16/0495; A61M 16/1005; A61M 16/0875; A61M 2210/0643; A61M 2210/0637; A61M 2202/0208
USPC 128/847; 128/848

(57) ABSTRACT

A dental oral appliance to open the airway for a sleeping individual who suffers with snoring or obstructive sleep apnea comprising, a body, structure configured to removably affix the appliance to the upper or lower teeth, structure to prevent occlusion of the upper and lower teeth, a transpalatal member that extends from the lingual of the right molars to the lingual of the left molars, a posterior tongue restrainer attached to the transpalatal member, and one or more tubes extending from the anterior of the appliance to the posterior of the appliance for the delivery of oxygen into the oropharyngeal airway.

### CROSS-REFERENCE TO RELATED APPLICATIONS

This application claims the benefit of U.S. Provisional Application No. 61/607,720, filed Mar. 7, 2012, the disclosure of which is incorporated herein by reference in its entirety.

### TECHNICAL FIELD

This invention is a dental oral appliance for use by patients who suffer with sleep disorders, to reduce or eliminate snoring and to open the airway for a sleeping individual who suffers with obstructive sleep apnea and to deliver oxygen to the open airway.

### BACKGROUND

It has been estimated that ninety million American adults and children snore and that three in every ten adults snores. Snoring can have serious medical consequences for some people. Snoring is the first indication of a potentially life-threatening sleep disorder called Obstructive Sleep Apnea. If not diagnosed or if left untreated, Obstructive Sleep Apnea could result in severe medical consequences such as systemic high blood pressure, cardiovascular disease and even sudden death.

Obstructive sleep apnea occurs during sleep when the tongue falls and rolls upward and backward, blocking the airway for 10-90 seconds. These events are measured by spending the night sleeping in a center which measures the number of air blockage events per hour. Less than 5 events per hour is normal. 5-19 events per hour is mild sleep apnea. 20-39 events per hour is moderate sleep apnea. Over 40 events per hour is severe sleep apnea.

For sleep apnea there are three main treatments of choice: the CPAP machine, surgery and oral sleep appliances. They are all designed to open the airway during sleep so that there is minimal or no air blockage.

Snoring is caused by vibration of the tissues due to air turbulence as the airway narrows and may be a sign that a patient is suffering from apnea. But not all snorers suffer from apnea. Snoring can be categorized by its severity. There is the snorer who snores but experiences no physical problems. Then, there is the snorer who suffers from apnea, or the snorer who suffers from upper airway resistance. In some of these people, though they may not actually experience 40 apneic episodes, their snoring is so loud and their breathing so labored, that it still wakes them, and their partners, numerous times throughout the night.

Many spouses, partners and/or children suffer through the night from the annoying noise of the snorer. Snoring not only disturbs the sleeping pattern of the snorer himself, it is also disruptive to the family life by causing lack of sleep to all involved. This leaves all involved unrefreshed, tired and sleepy throughout the day. It can cause sleepiness while driving, reading, working or doing other tasks.

A broad variety of intra-oral and dental appliances and devices are now available to treat a patient for snoring. Some known oral devices for treating snoring and obstructive sleep apnea are worn inside of the mouth and work by repositioning of the jaw, moving the mandible, lifting the soft palate or moving the tongue forward. These appliances work by advancing the tongue and soft palate away from the back wall of the throat. These devices are often quite uncomfortable.

Other methods used to treat snoring include controlled positive air-flow pressure systems, also known as CPAP, which require a nose mask and which are quite uncomfortable.

Still other treatments for snoring include various surgeries, which are drastic steps to take to attempt to cure the problem, however snoring can be so disruptive to a person's life and relationships, that some sufferers resort to surgery.

### SUMMARY OF THE INVENTION

The sleep appliance of this invention is a new design which encompasses many of the features of the following previous sleep appliance patents. Incorporated by reference herein are the following U.S. Patents; U.S. Pat. No. 6,766,802, issued Jul. 27, 2004; U.S. Pat. No. 7,451,767, issued Nov. 18, 2008; U.S. Pat. No. 7,861,722, issued Jan. 4, 2011; U.S. Pat. No. 7,861,724, issued Jan. 4, 2011; and U.S. Pat. No. 8,132,567, issued Mar. 13, 2012. The improved design of this invention comprises adding one or more tubes that extend from the front, anterior, of the appliance to the back, posterior. The front end of the tubes are connected to an oxygen supply, or a machine that will deliver oxygen through the tubes, into the open oropharyngeal airway, created by the sleep appliance.

This sleep appliance has a posterior tongue restrainer, or tail, as described in the earlier patents cited above, especially U.S. Pat. No. 8,132,567. There may be one tail or a plurality of tails that can be used to further open the oropharyngeal airway.

This new sleep appliance can have a posterior tongue restrainer which does not depress the tongue but does inhibit the upward and backward movement of the tongue. Alternatively, the posterior tongue restrainer can depress the tongue. The choice depends upon the needs of the patient based upon whether they require depression of the tongue for effective results. The goal of this appliance is to open the airway and deliver oxygen to the open airway.

This new sleep appliance fits over either the upper or lower arch (teeth). It may utilize the body design disclosed in any of the referenced issued patents. The appliance has a bite discluder in the anterior or posterior position to disclude or separate the upper and lower teeth. The bite is opened 1 mm or more in the anterior teeth area.

A transpalatal, translingual bar follows the designs of the cited issued patents. The bar extends from the lingual of the upper or lower right molars, to the lingual of the upper or lower left molars. The transpalatal bar can be straight or curved.

The posterior tongue restrainer can also follow all of the designs disclosed in the cited patents. There may be one posterior tongue restrainer or a plurality of posterior tongue restrainers extending back from the transpalatal bar, depending upon the needs of the patient. The posterior tongue restrainers can be from 1 mm in length to 2 inches or more in length. The posterior tongue restrainers can be placed parallel to the occlusal plane or can be angled down from the horizontal, 5 degrees to 80 degrees toward the tongue, depending upon the needs of the patient. This creates the restraining and depression effect to keep the airway open at night.

The posterior tongue restrainer (tail) can also be dropped into a horizontal position, parallel to the occlusal plane, from ⅛ of an inch to as far as ¾ of an inch. In this case, it is stepped down from the transpalatal bar and is “L” shaped.

On the tongue (lingual) side of either an upper or lower sleep appliance is a tube of approximately 0.5 mm, or more, in circumference, extending from about 2 inches to about 12 inches or more, preferably about 6 inches, anterior of the lateral incisors, along either one or both sides of the appliance, to as far back as the posterior extension of the body of the appliance, on both the right and left side. The tubes can be placed above, below or through the transpalatal, translingual bar. The tubes can be short of the posterior tongue restrainer (tail) extension of the appliance. The tubes are for the delivery of oxygen. The tubes can be constructed of any material that is compatible with the environment of the mouth, preferably plastic.

In addition to the basic design of two tubes of oxygen delivery, there can be only one tube, or a plurality of tubes, as many tubes as are required to provide sufficient oxygen for the patient. The addition of feeding oxygen to the sleeping patient, through the sleep appliance worn by the patient, makes his or her sleep more comfortable by reducing or eliminating snoring or sleep apnea.

The tubes for the upper appliance, for instance, are on the lingual (tongue) side of the appliance, placed anywhere from lmm above the occlusal table, to as high up as the highest extension of the acrylic appliance, as it fits against the palate. The tubes are held in place by any one of a plurality of mechanisms that will keep the tubes attached to the body of the appliance. The tube can be completely or partially encased in plastic as it travels back from the lateral incisors to its most posterior extension, at the posterior of the appliance.

The tubes of the lower appliance are on the lingual (tongue) side of the appliance, placed anywhere from lmm below the occlusal table, to as low as the lowest extension of the acrylic, as it fits against the lingual walls of the mandible. The tubes are held in place by any one of a plurality of mechanisms that will keep the tubes attached to the appliance. The tubes can be completely or partially encased in plastic as they travel back from the lateral incisors to their most posterior extension at the posterior of the appliance.

### BRIEF DESCRIPTION OF THE DRAWINGS

FIG. 1 is a maxillary, biting surface view, of the appliance of this invention;

FIG. 2 is a biting surface view, showing the air tubes extending to the posterior of the appliance;

FIG. 3 is a rear view showing the posterior end of the air tubes;

FIG. 4 is a view from the underside, the side that fits over the teeth for retention, showing a posterior tongue restrainer, horizontal and parallel to the occlusal plane surface on which upper and lower teeth mesh.

FIG. 5 is a side view of an appliance with a posterior tongue restrainer angled down toward the tongue at about 20-30 degrees.

FIG. 6 is a side view of an appliance a posterior tongue restrainer angled down toward the tongue at about 60-70 degrees.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

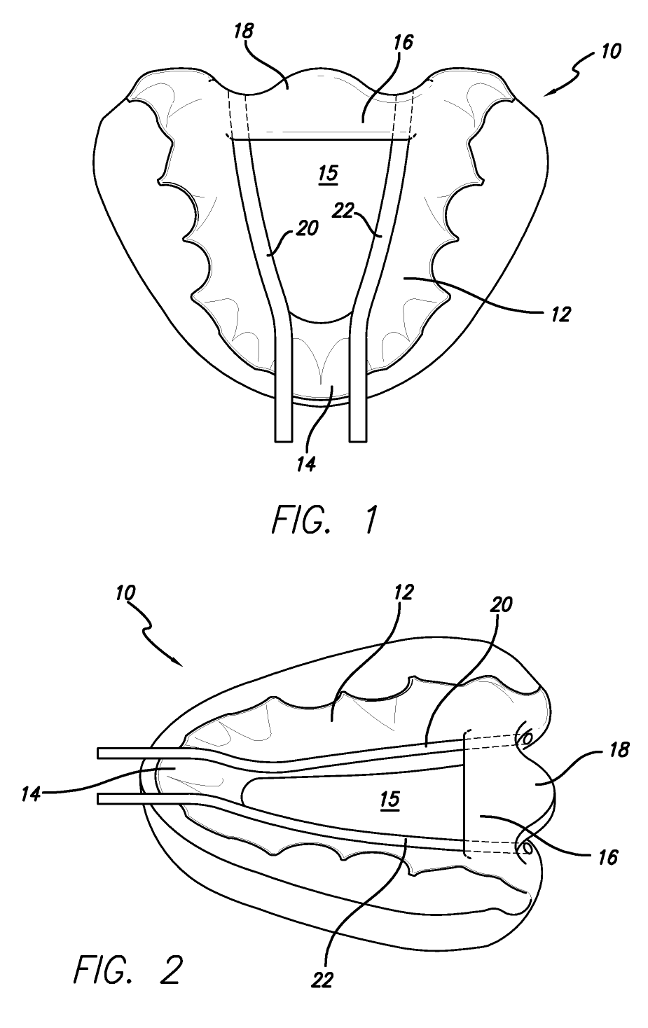

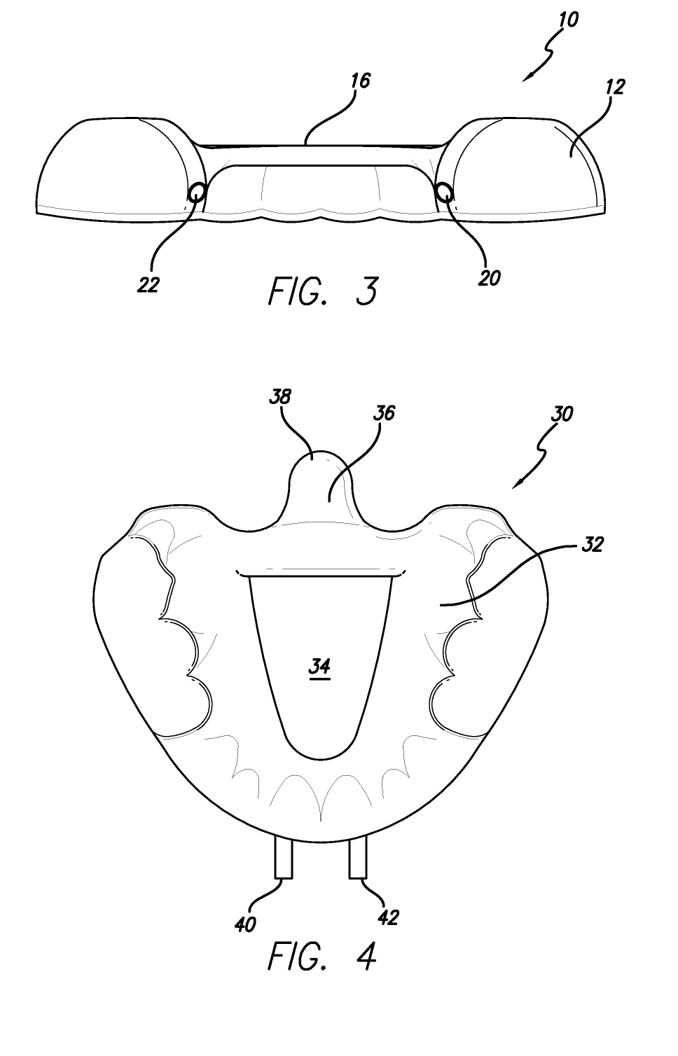

Referring now to the drawings, there is shown in FIGS. 1-3, a sleep appliance 10 of the present invention which comprises a body portion 12 which fits adjacent the inside of the upper or lower teeth of the person wearing it. Body 12 is generally U-shaped with an open palate 15 and is custom fitted to fit over the wearer's posterior upper or lower teeth, in the same manner as an occlusal night guard, which uses an occlusal coverage. The occlusal coverage holds appliance 10 firmly onto the posterior teeth. The appliance 10 is made using an acrylic plastic commonly used for dental appliances. It can be fitted with various retentive dental clasps to increase the hold (retention) of the appliance.

Bilaterally from both cuspids back, the material on the occlusal table is elevated to create an opening separation in the anterior sector 14 of the appliance. The posterior occlusal table is elevated based on need of strength of material and space needed in the anterior portion 14 of the appliance. The anterior space created will allow space for the air tubes to pass through from the appliance to the oxygen supply.

A transpalatal bar 16 extends from the right side to the left side of appliance 10, inhibiting the upward and backward movement of the tongue to keep the airway open during sleep.

A posterior tongue restrainer 18 is attached to, or molded as part of, the center rear portion of the transpalatal bar 16, and extends rearward. In this embodiment, the posterior tongue restrainer 18 can or cannot depress the tongue depending upon that which needs to be done to open the airway. A plurality of posterior tongue restrainers can be attached to, or molded as part of the transpalatal bar, if required for the patient.

A pair of tubes 20 and 22 are on the lingual (tongue) side of either an upper or lower sleep appliance. Tubes 20 and 22 have a circumference of about 0.5 mm, or more, and extend anterior of the lateral incisors, along either or both sides of the appliance, to as far back as the posterior extension of the body of the appliance, on both the right and left side. Tubes 20 and 22 can pass either above, below or through transpalatal bar 16 and can extend short of the posterior tongue restrainer (tail) extension 18 of the appliance. Tubes 20 and 22 are for the delivery of an adequate supply of oxygen in the amounts of about 19% to about 24% percent oxygen in air, to allow for maximum cell health. The tubes can be constructed of any material that is compatible with the environment of the mouth, preferably plastic.

Tubes 20 and 22 can be fastened to body 12 of appliance 10 by the use of acrylic plastic covering some or all of the tubes. Tubes 20 and 22 can extend as far as 12 inches or more anterior of the lateral incisors, and will be connected to the supply of oxygen which will be fed through the tubes to the wearer's oropharyngeal airway.

While two air tubes are shown in the drawings described, there may be only one tube or a plurality of tubes connected to the appliance 10, depending upon the needs of the patient.

Referring to FIG. 4, there is shown a sleep appliance 30 comprising a body 32 with an open palate 34. An occlusal coverage holds appliance 30 firmly onto the posterior teeth. Appliance 30 is made using an acrylic plastic commonly used for dental appliances.

A transpalatal bar 36 extends from the right side to the left side of appliance 30, inhibiting the upward and backward movement of the tongue to keep the airway open during sleep. Transpalatal bar 36 can be configured to depress the tongue if needed to keep the airway open during sleep, depending upon the needs of the patient.

A posterior tongue restrainer (tail) 38 is attached to, or molded as part of, the center rear portion of the transpalatal bar 36, and extends rearward to further inhibit the upward and backward movement of the tongue. Tail 38 extends backward from transpalatal bar 36 horizontal and parallel to the occlusal plane. In this embodiment there is no depression of the tongue, however the tail inhibits the upward and backward movement of the tongue.

Oxygen tubes 40 and 42 extend from the anterior of the appliance 30, to which will be connected the oxygen supply.

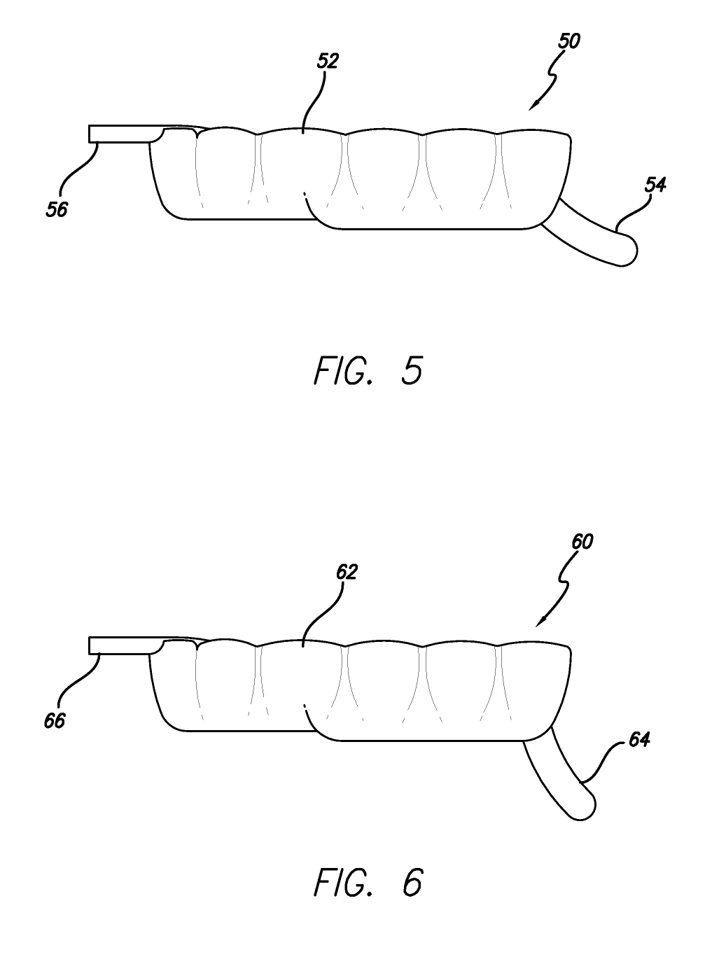

Referring to FIG. 5, there is shown a side view of a sleep appliance 50 having a body 52, a posterior tongue restrainer (tail) 54 and a tube 56. Tail 54 is angled down toward the tongue at about 20-30 degrees and depresses the tongue to open the airway of the patient.

Referring to FIG. 6, there is shown a side view of a sleep appliance 60 having a body 62, a posterior tongue restrainer (tail) 64 and a tube 66. Tail 64 is angled down toward the tongue at about 60-70 degrees and depresses the tongue to open the airway of the patient.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A dental oral appliance to open the airway for a sleeping individual who suffers with snoring or sleep apnea comprising, a body, structure configured to removably affix the appliance to the upper or lower teeth, structure to prevent occlusion of the upper and lower teeth, a transpalatal member that extends from the lingual of the right molars to the lingual of the left molars, one or more posterior tongue restrainers attached to the transpalatal member, and one or more tubes extending from the anterior of the appliance to the posterior of the appliance.

2. The dental oral appliance of claim 1 in which the body has an open palate.

3. The dental oral appliance of claim 1 in which the one or more posterior tongue restrainers are straight or curved.

4. The dental oral appliance of claim 1 in which the transpalatal bar is straight or curved.

5. The dental oral appliance of claim 1 in which the tubes are attached to the body of the appliance.

6. The dental oral appliance of claim 5 in which the tubes are attached to the body by acrylic plastic.

7. The dental oral appliance of claim 1 in which a tongue restrainer is angled down towards the tongue.

8. The dental oral appliance of claim 7 in which a tongue restrainer is angled down towards the tongue 20 to 30 degrees.

9. The dental oral appliance of claim 7 in which a tongue restrainer is angled down towards the tongue 60 to 70 degrees.

10. The dental oral appliance of claim 1 in which the tubes are attached to a supply of oxygen.

11. A dental oral appliance to open the airway for a sleeping individual who suffers with snoring or sleep apnea comprising, a body, means to removably affix the appliance to the upper or lower teeth, means to prevent occlusion of the upper and lower teeth, a transpalatal member that extends from the lingual of the right molars to the lingual of the left molars, one or more posterior tongue restrainers attached to the transpalatal member, and one or more tubes extending from the anterior of the appliance to the posterior of the appliance, the tubes adapted to enable the flow of oxygen to the airway of the sleeping individual.

12. The dental oral appliance of claim 11 in which the body has an open palate.

13. The dental oral appliance of claim 11 in which the one or more posterior tongue restrainers are straight or curved.

14. The dental oral appliance of claim 11 in which the transpalatal bar is straight or curved.

15. The dental oral appliance of claim 11 in which the tubes are attached to the body of the appliance.

16. The dental oral appliance of claim 15 in which the tubes are attached to the body by acrylic plastic.

17. The dental oral appliance of claim 11 in which a tongue restrainer is angled down toward the tongue.

18. The dental oral appliance of claim 17 in which a tongue restrainer is angled down towards the tongue 20 to 30 degrees.

19. The dental oral appliance of claim 17 in which a tongue restrainer is angled down towards the tongue 60 to 70 degrees.

20. The dental oral appliance of claim 11 in which the tubes are attached to a supply of oxygen.
